(19) United States
(12) Patent Application Publication
(10) Pub. No.: US 2026/0263747 A1
(43) Pub. Date: Sep. 10, 2026

(54) DEVICE FOR PROLONGED DRAIN SECUREMENT

(71) Applicants: Ahsun RIAZ, Evanston, IL (US); Abhinav TALWAR, Evanston, IL (US); Riad SALEM, Evanston, IL (US); Northwestern University, Evanston, IL (US)

(72) Inventors: Ahsun RIAZ, Evanston, IL (US); Abhinav TALWAR, Evanston, IL (US); Riad SALEM, Evanston, IL (US)

(21) Appl. No.: 18/993,463

(22) PCT Filed: Jul. 13, 2023
(86) PCT No.: PCT/US23/70143

§ 371 Date: Jan. 10, 2025

Related U.S. Application Data
(60) Provisional application No. 63/388,919, filed on Jul. 13, 2022.

(51) Int. Cl. A61M 25/02

(52) U.S. Cl. CPC A61M 25/02; A61M 2025/024; A61M 2025/0286

(57) ABSTRACT

Provided herein are devices for prolonged securement of medical devices to a subject. In particular, drain tubes (e.g., percutaneous drains), catheters, and other medical devices are secured near the insertion site using the devices herein.

### CROSS-REFERENCE TO RELATED APPLICATIONS

This application claims the benefit of U.S. Provisional Patent Application No. 63/388,919, filed on Jul. 13, 2022, which is incorporated by reference herein.

### FIELD

Provided herein are devices for prolonged securement of medical devices to a subject. In particular, drain tubes (e.g., percutaneous drains), catheters, and other medical devices are secured near the insertion site using the devices herein.

### BACKGROUND

Clinicians, for example, interventional radiologists (IR) and surgeons, place drains and other catheters percutaneously as conduits for fluid to be removed from bodily cavities. Many of these drains are removed within a few weeks of initial placement. However, there are drains that remain for prolonged periods (more than 2 months). A growing body of evidence indicates that complications of drain placement—pain, leakage, dislodgment, and infection—occur frequently. Many of these complications are attributable, at least in part, to the methods used to place and secure drains. Suture is often used to tie drains to skin, but loose knots predispose to drain dislodgment while tight knots frequently cause pain and serve as niduses for infection. Adhesive drain fixation devices lose efficacy over time and make dressing changes unnecessarily complicated. What is needed is a device that allows for stable and secure attachment of drain/catheter devices for prolonged periods of time, while allowing for patient movement, dressing changes and other activities, without producing discomfort of adverse events.

### SUMMARY

Provided herein are devices for prolonged securement of medical devices to a subject. In particular, drain tubes (e.g., percutaneous drains), catheters, and other medical devices are secured near the insertion site using the devices herein.

In some embodiments, provided herein are devices for securing a medical line to a subject, the device comprising: (a) a fastener comprising (i) a band capable of encircling the line cross-sectionally and having teeth formed on one side of the band, and (ii) a first receptacle comprising a first opening capable of receiving the band lengthwise and a first pawl configured to engage with the teeth of the band and to lock the fastener around the line; (b) a mounting plate comprising a front face and a rear face; (c) a second receptacle mounted the mounting plate, comprising a second opening capable of receiving the band lengthwise and a second pawl configured to engage with the teeth of the band and to couple the fastener to the mounting plate; (d) an aperture through the mounting plate to accommodate the medical line passing through the device from the rear to front; and (e) a chemical or mechanical securement element on the rear face of the mounting plate and configured to secure the mounting plate to the subject. In some embodiments, the fastener is a zip tie. In some embodiments, the first pawl and the teeth of the band are configured to interact to maintain the grip of the fastener around the medical line. In some embodiments, the first pawl and the teeth of the band are configured to interact to prevent backsliding and/or removal of the band from the receptacle. In some embodiments, the device comprises one or more protrusions and/or channels on the front face of the mounting plate. In some embodiments, the protrusions and/or channels define locations for positioning the medical line and/or the band on the front face of the mounting plate. In some embodiments, the mounting plate comprise an open channel running vertically along the front face of mounting plate for positioning the medical line. In some embodiments, the aperture comprises a funnel shape with a wider diameter toward the front face of the mounting plate. In some embodiments, the chemical securement element is a physiologically-compatible adhesive material (e.g., a medical-grade glue). In some embodiments, the device further comprises a removable film cover over the adhesive material. In some embodiments, the mechanical securement element is one or more screws or barbed microneedles configure to reside in the subcutaneous tissue when the device is placed on the skin of a subject.

In some embodiments, provided herein are systems comprising devices for securing medical lines herein and a medical line. In some embodiments, the medical line is a percutaneous drain or a catheter. In some embodiments, the medical line extends from an opening in the subject through the aperture, through the fastener and along the channel of the mounting plate. In some embodiments, the mounting plate is adhered to the skin of the subject such that the aperture is placed over the opening in the subject, wherein the fastener is securely tightened around the medical line, and wherein the fastener is coupled to the second receptacle.

In some embodiments, provided herein are methods of securing a medical line to a subject using the device herein, the methods comprising: (a) securing the rear face of the mounting plate of the device to the subject via the securement element, wherein the aperture of the mounting plate is aligned with an opening in the subject; (b) placing the medical line through the aperture and into the opening in a subject; (c) tightening the fastener around the medical line; and (d) coupling the fastener to the second receptacle, thereby securing the medical line in place on the subject. In some embodiments, the medical line is a percutaneous drain or a catheter.

### BRIEF DESCRIPTION OF THE DRAWINGS

FIG. 1. Drawing of a percutaneous cholecystostomy (PC) drain (black arrow) which is a drain placed through the skin (red arrow) to the gallbladder (blue arrow). Such PC drains are an exemplary application of the devices herein.

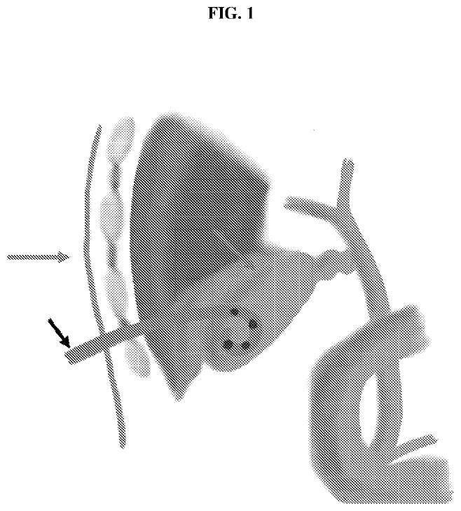

FIG. 2. Drawing of a front-face view of an exemplary device embodiment.

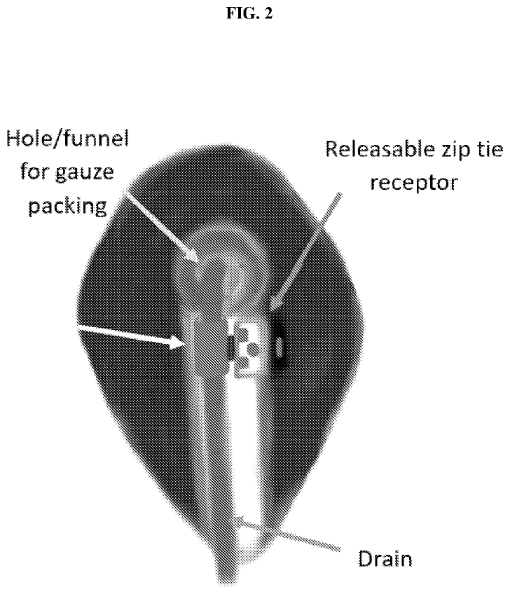

FIG. 3. 3D rendering of a front-face view of an exemplary device embodiment.

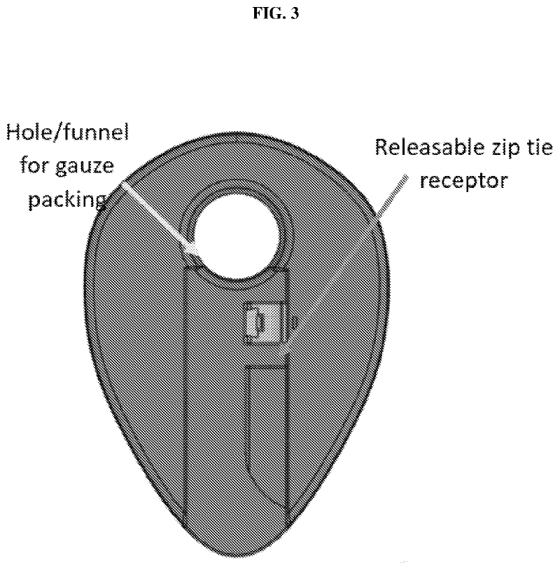

FIG. 4. Drawing of a side view of an exemplary device embodiment.

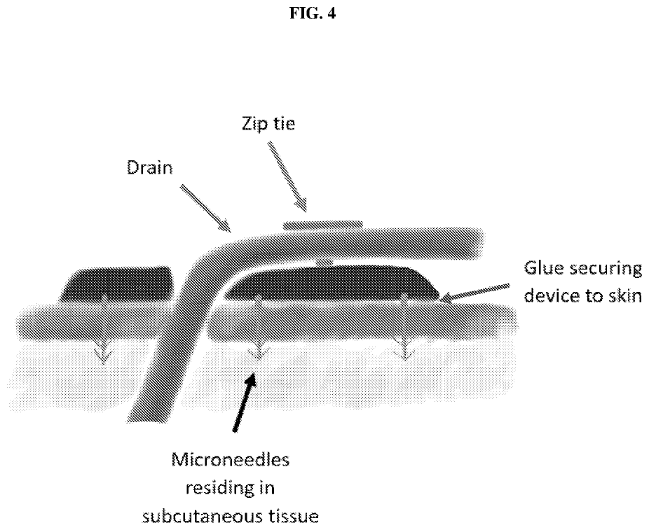

FIG. 5. 3D rendering of a side view of an exemplary device embodiment.

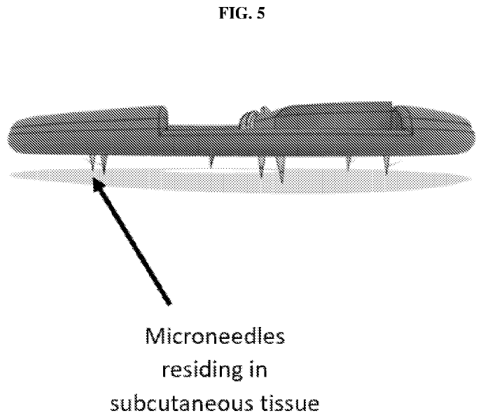

FIG. 6. Drawing of a top view of an exemplary device embodiment.

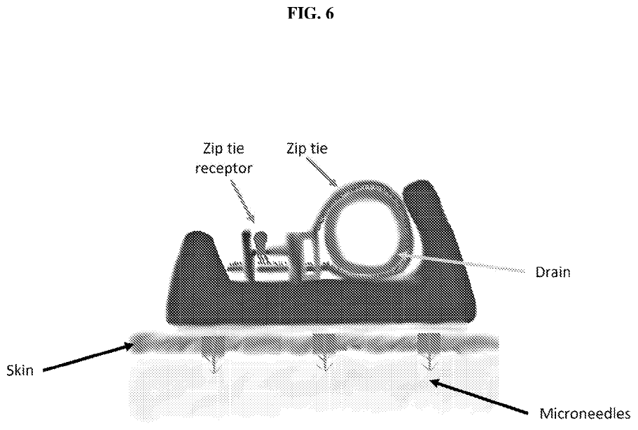

### DEFIITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

### DETAILED DESCRIPTION

Provided herein are devices for prolonged securement of medical devices to a subject. In particular, drain tubes (e.g., percutaneous drains), catheters, and other medical devices are secured near the insertion site using the devices herein.

Provided herein are medical devices that function to fix a medical line (e.g., catheter, percutaneous drain, IV tubing, etc.) to a patient. In typical embodiments herein, the medical line is to be fixed to the subject for a prolonged time period (e.g., 2 months or greater (e.g., 2 months, 3 months, 4 months, 5 months, 6 months, 9 months, 1 year, 2 years, or more, or ranges therebetween). In some embodiments, the devices herein are well-suited for prolonged device securement. In some embodiments, the devices herein stabilize and position a medical line without the use of a suture. In some embodiments, the devices herein allow the medical line to be stably held in place with respect to the point of entry into the subject (e.g., an incision or orifice). In some embodiments, a device allows the patient to move while the line is secured by the device. In some embodiments, a device allows the position of the body of the medical line (not the insertion stie) to be adjusted while the line is secured by the device. Certain embodiments are described herein for use with percutaneous drains; however, the embodiments described herein may also find use in the sutureless securement of other medical lines, such as catheters (e.g., urinary catheters) and IV tubing. Embodiments herein are not limited by the identity of the medical lines to be used therewith.

In some embodiments, the devices herein are drain fixation devices for prolonged use that are more comfortable for patients, easier to exchange during dressing changes, and decrease the incidence of drain-related adverse events.

In particular embodiments, provided herein are device for securing a medical line (e.g., tube) at an incision site of a patient for a prolonged time period. In some embodiments, the medical line is a percutaneous drain. Exemplary medical lines that find use with embodiments of the devices herein may include an endotracheal tube, a tracheotomy tube, chest tube, a vascular catheter, an urinary catheter, a nephrostomy tube, a biliary stent, a peritoneal catheter, an epidural catheter, a central nervous system catheter, a prosthetic valve, a gastric tube, an intestinal tube, drug-delivery implant, or an intravenous tube. In some embodiments, the devices herein find use in securing any medical line that enters the subject (e.g., at an incision or through an orifice) and needs to be held in place on the skin of the subject.

In some embodiments, devices herein comprise a fastener element. In some embodiments, the fastener engages with and secures the medical line. In some embodiments, the fastener wraps circumferentially around the medical line, such that the medical line extends through the closed fastener. In some embodiments, the fastener forms a loop around the medical line. In some embodiments, tightening of the loop around the medical line couples the fastener to the medical line.

In some embodiments, the fastener element comprises a band and a receptacle (e.g., first receptable) capable of receiving the band and securely engaging along the length of the band.

In some embodiments, the fastener comprises an elongated band with a width of 1-15 mm (e.g., 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or ranges therebetween) and a length of 5-20 cm (e.g., 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, or ranges therebetween). In some embodiments, the band is substantially flat, having a depth that is less than its width (e.g., less than 5 mm (e.g., 4 mm, 3 mm, 2 mm, 1 mm, or less or ranges therebetween). In some embodiments, the band comprises teeth, grooves, indentations, protrusions, etc. disposed along its length (e.g., regularly spaced along all or a portion of the length of the band). In some embodiments, the teeth, grooves, indentations, protrusions, etc. are presented on one or both sides of the band.

In some embodiments, the receptacle (e.g., first receptable) comprises a housing with an opening through the housing configured to accept the band of the fastener. In some embodiments, the receptacle (e.g., first receptable) is positioned at one terminus of the band. In some embodiments, the opening in the receptacle (e.g., first receptable) is sized to accommodate the cross-sectional shape of the band. In some embodiments, the receptacle (e.g., first receptable) comprises a band-engagement element configured to engage with the teeth, grooves, indentations, protrusions, etc. disposed along the band. In some embodiments, the band-engagement element is a pawl. In some embodiments, the pawl allows the teeth, grooves, indentations, protrusions, etc. of the band to pass through the opening in the receptacle housing in a first direction, but will not allow the band to be removed from the receptacle (e.g., first receptable) in the opposite direction (e.g., no backsliding of the band).

In some embodiments, the fastener comprises a tightening mechanism configured to tighten the grip of the fastener around the medical line. The tightening mechanism may comprise manually pulling the band through the opening of the receptacle (e.g., first receptable). In some embodiments, the receptacle (e.g., first receptable) is a locking receptacle. Once the band is advanced into the opening, teeth, grooves, indentations, protrusions, etc. on the band interact with a mechanism in the receptacle (e.g., a pawl) to prevent removal (e.g., via backsliding) of the band from the receptacle. In some embodiments, the tightening mechanism utilizes the pawl of the first receptacle to advance the band step-wise (e.g., tooth by tooth) into and through the receptacle. In some embodiments, the tightening mechanism is in the form of a rotatable knob and a rod passing through the receptacle (e.g., first receptable), such that rotation or pressing of the knob results in the rod advancing the band into and through the receptacle. In some embodiments, the fastener utilizes a worm-gear mechanism to advance the band through the receptacle (e.g., first receptable), to tighten the grip on the medical line, and/or to prevent backsliding.

In some embodiments, the fastener utilizes a “zip-tie” mechanism. Zip-ties will be familiar to the skilled artisan. The band of the zip tie forms a loop around the medical line and is then inserted through the receptacle of the zip tie. In some embodiments, the receptacle (e.g., first receptable) of the zip tie is an opening configured to receive the band. The receptacle (e.g., first receptable) comprises a pawl or similar mechanism to engage with teeth on the band. The pawl and teeth interact when the band is inserted through the opening of the receptacle (e.g., first receptable) to prevent the band from backsliding through the receptacle and loosening the grip of the fastener on the medical line. In some embodiments, the receptacle is located on one end of the fastener.

In some implementations, the fastener (e.g., zip-tie) is provided unassembled, i.e., such that upon positioning medical line on the subject, the user inserts the toothed band into the locking receptable, thus encircling the medical line, and then advancing the band through the first receptacle to tighten the grip of the fastener around the medical line. In other implementations, the fastener (e.g., zip tie) is pre-assembled, i.e., with the band already inserted, to a certain extent, through the receptacle. In such cases, the medical line is fed through the loop in the band and then the band is further advanced into the receptacle to tighten the grip on the medical line.

In some embodiments, devices herein comprise a mounting plate. In some embodiments, the mounting plate is configured to be placed on the skin of the subject, on or adjacent to the insertion stie of the medical line into the subject. In some embodiments, the mounting place comprises a front face configured to face away from the skin surface of the subject when the device is positioned on the subject. In some embodiments, the mounting place comprises a rear face configured to contact the skin of the subject when the device is positioned on the subject.

In some embodiments, the mounting plate is placed on the skin of the subject at the location of the insertion stie of the medical line into the subject. In some embodiments, the mounting plate comprises an aperture through the mounting plate that is configured to receive the medical line and allow the medical line to pass from the rear of the device to the front of the device. In some embodiments, the aperture is of sufficient diameter to allow the medical line to pass through. In some embodiments, the aperture is of sufficient diameter to allow the mounting plate to sit on the skin of the subject, surrounding the opening in the subject (e.g., incision) without the mounting plate contacting the opening. In some embodiments, the aperture has a diameter of 2-20 mm (e.g., 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, or ranges therebetween). In some embodiments, the aperture is funnel shaped, having a wider opening on the front face of the mounting plate than the opening on the rear face of the mounting plate.

In some embodiments, the front face of the mounting plate comprises one or more elements configured to provide a path for the medical line to traverse along the front face of the mounting plate. In some embodiments, the medical line is configured to reside within an open-face channel along the front face of the mounting plate when properly positioned. In some embodiments, one or more recesses and/or protrusions guide the medical line into proper position on the front face of the mounting plate. In some embodiments, the medical line is free to move and reposition on the front face of the mounting plate. In some embodiments, the front face of the mounting plate comprises one or more clips or other securement elements to hold the medical line in place on the front face of the mounting plate.

In some embodiments, the rear face of the mounting plate comprises a securement element for fixing the mounting plate to the skin of a subject. In some embodiments, the rear face comprises a chemical securement element. In some embodiments, a chemical securement element is an adhesive material that is present on or can be applied to the rear face of the mounting plate (or can be applied to the skin of the subject) and serves to secure the mounting plate in place on the skin of the subject. In some embodiments, the adhesive material is a glue or other adhesive. In some embodiments, the adhesive is medical grade and/or biocompatible. In some embodiments, the adhesive is synthetic or biological in origin. Suitable adhesive will be understood in the field. In some embodiments, the rear face comprises a mechanical securement element. In some embodiments, a mechanical securement element is structure that protrudes from the rear face of the mounting plate and secures the device to the skin of the subject. In some embodiments, the mechanical securement element protrudes into the skin or subcutaneous tissue of the subject. In some embodiments, a mechanical securement element is one or more microneedles, screws, pins, etc. In some embodiments, a mechanical securement element is one or more microneedles comprising barbs or other protrusions to secure the microneedles within the subcutaneous tissue of the subject. In some embodiments, a mounting plate is secured to the skin of a subject by a combination of adhesive and microneedles.

In some embodiments, a device herein comprises a receptable (e.g., a second receptacle), coupled to the mounting plate, and capable of receiving the band of the fastener. In some embodiments, the mounting plate receptable (e.g., a second receptacle) is attached to the front face of the mounting plate. In some embodiments, the receptable (e.g., a second receptacle) resides adjacent to the aperture. In some embodiments, the receptable (e.g., a second receptacle) resides adjacent to the intended pathway for the medical line across the face of the mounting plate (e.g., adjacent to the channel). In some embodiments, the receptable (e.g., a second receptacle) is of substantially the same structure and mechanism as the receptable (e.g., a first receptacle) on the fastener. In some embodiments, the receptable (e.g., a second receptacle) comprises a distinct structure and/or mechanism as the receptable (e.g., a first receptacle) on the fastener.

In some embodiments, the receptacle (e.g., a second receptacle) comprises a housing with an opening through the housing configured to accept the band of the fastener. In some embodiments, the opening in the receptacle (e.g., a second receptacle) is sized to accommodate the cross-sectional shape of the band. In some embodiments, the receptacle comprises a band-engagement element configured to engage with the teeth, grooves, indentations, protrusions, etc. disposed along the band. In some embodiments, the band-engagement element is a pawl. In some embodiments, the pawl allows the teeth, grooves, indentations, protrusions, etc. of the band to pass through the opening in the receptacle housing in a first direction, but will not allow the band to be removed from the receptable in the opposite direction (e.g., no backsliding of the band). In some embodiments, insertion of the band into the receptacle (e.g., second receptacle) secures the fastener to the mounting plate and properly positions the medical line on the device and relative to the point of entry into the subject (e.g., incision).

In some embodiments, the receptacle (e.g., a second receptacle) comprises a mechanism configured to advance the receptacle (e.g., a second receptacle) along the band. The mechanism may comprise manually pulling the band through the opening of the receptacle (e.g., a second receptacle). In some embodiments, the receptacle is a locking receptacle. Once the band is advanced into the opening, teeth, grooves, indentations, protrusions, etc. on the band interact with a mechanism in the receptacle (e.g., a pawl) to prevent removal (e.g., via backsliding) of the band from the receptacle. In some embodiments, the tightening mechanism utilizes the pawl of the first receptacle to advance the band step-wise (e.g., tooth by tooth) into and through the receptacle. In some embodiments, the mechanism is in the form of a rotatable knob and a rod passing through the receptacle, such that rotation or pressing of the knob results in the rod pressing advancing the band into and through the receptacle. In some embodiments, the receptacle (e.g., a second receptacle) utilizes a worm-gear mechanism to advance the band through the receptacle and/or to prevent backsliding. In some embodiments, the mechanism for advancing the band into the receptacle (e.g., a second receptacle) and/or for locking the band within the receptacle (e.g., a second receptacle) is releasable. In some embodiments, release of the mechanism allows the fastener and/or the medical line encircled therewith to be disengaged from the mounting plate.

In some embodiments, the receptacle (e.g., a second receptacle) comprises an opening configured to receive the band. The receptacle (e.g., a second receptacle) comprises a pawl or similar mechanism to engage with teeth on the band. The pawl and teeth interact when the band is inserted through the opening of the receptacle (e.g., a second receptacle) to prevent the band from backsliding through the receptacle and uncoupling with the mounting plate.

In some embodiments, the devices herein are intended for use with medical lines that are to be used for a period of time of two months or longer (e.g., 2 months, 3 months, 4 months, 5 months, 6 months, 9 months, 1 year, 2 years, or more, or ranges therebetween). In some embodiments, the devices herein are intended for use with medical lines that do not require frequent, routine scheduled changes.

In some embodiments, devices herein and components there of comprises any suitable materials. Suitable materials include plastics and metals.

In some embodiments, provided herein are methods of securing a medical line to a subject using a device herein. In some embodiments, methods comprise one or more of the steps of (a) securing the rear face of the mounting plate of the device to the subject via the securement element, wherein the aperture of the mounting plate is aligned with an opening in the subject; (b) placing the medical line through the aperture and into the opening in a subject; (c) tightening the fastener around the medical line; and (d) coupling the fastener to the second receptacle, thereby securing the medical line in place on the subject. In some embodiments, the mounting plate is secured to the subject before the medical line is inserted. In some embodiments, the mounting plate is secured to the subject after the medical line is inserted.

In some embodiments, provided herein is a system comprising a medical line and a device for securing the medical line to a subject for a prolonged period of time.

1. A device for securing a medical line to a subject, the device comprising:
(a) a fastener comprising (i) a band capable of encircling the line cross-sectionally and having teeth formed on one side of the band, and (ii) a first receptacle comprising a first opening capable of receiving the band lengthwise and a first pawl configured to engage with the teeth of the band and to lock the fastener around the line;
(b) a mounting plate comprising a front face and a rear face;
(c) a second receptacle mounted the mounting plate, comprising a second opening capable of receiving the band lengthwise and a second pawl configured to engage with the teeth of the band and to couple the fastener to the mounting plate;
(d) an aperture through the mounting plate to accommodate the medical line passing through the device from the rear to front; and
(e) a chemical or mechanical securement element on the rear face of the mounting plate and configured to secure the mounting plate to the subject.

2. The device of claim 1, wherein the fastener is a zip tie.

3. The device of claim 1, wherein the first pawl and the teeth of the band are configured to interact to maintain the grip of the fastener around the medical line.

4. The device of claim 1, wherein the first pawl and the teeth of the band are configured to interact to prevent backsliding and/or removal of the band from the first receptacle.

5. The device of claim 1, comprising one or more protrusions and/or channels on the front face of the mounting plate.

6. The device of claim 6, wherein the protrusions and/or channels define locations for positioning the medical line and/or the band on the front face of the mounting plate.

7. The device of claim 7, wherein the mounting plate comprises an open channel running vertically along the front face of mounting plate for positioning the medical line.

8. The device of claim 1, wherein the aperture comprises a funnel shape with a wider diameter toward the front face of the mounting plate.

9. The device of claim 8, wherein the chemical securement element is a physiologically-compatible adhesive material.

10. The device of claim 9, further comprising a removable film cover over the adhesive material.

11. The device of claim 8, wherein the mechanical securement element is one or more screws or barbed microneedles configure to reside in subcutaneous tissue of the subject when the device is placed on the skin of the subject.

12. A system comprising:
(a) the device of one of claims 1-11; and
(b) a medical line.

13. The system of claim 12, wherein the medical line is a percutaneous drain or a catheter.

14. The system of claim 12, wherein the medical line extends from an opening in the subject through the aperture, through the fastener and along the channel of the mounting plate.

15. The system of claim 14, wherein the mounting plate is adhered to the skin of the subject such that the aperture is placed over the opening in the subject, wherein the fastener is securely tightened around the medical line, and wherein the fastener is coupled to the second receptacle.

16. A method of securing a medical line to a subject using the device of one of claims 1-11, the method comprising:
(a) securing the rear face of the mounting plate of the device to the subject via the securement element, wherein the aperture of the mounting plate is aligned with an opening in the subject;
(b) placing the medical line through the aperture and into the opening in a subject;
(c) tightening the fastener around the medical line; and
(d) coupling the fastener to the second receptacle, thereby securing the medical line in place on the subject.

17. The method of claim 16, wherein the medical line is a percutaneous drain or a catheter.
